(19) United States
(12) Patent Application Publication
(10) Pub. No.: US 2023/0133195 A1
(43) Pub. Date: May 4, 2023

(54) GLUCOSE MONITORING OVER PHASES AND CORRESPONDING PHASED INFORMATION DISPLAY

(71) Applicant: DexCom, Inc., San Diego, CA (US)

(72) Inventors: Alexander Michael Diener, San Diego, CA (US); Stacey Lynne Fischer, San Diego, CA (US); Harry Shaw Strothers, San Diego, CA (US); Chad M. Patterson, San Diego, CA (US); Justin Yuen, San Diego, CA (US); Apurv U. Kamath, San Diego, CA (US); Andrew Merrill Terry, San Diego, CA (US); Margaret A. Crawford, San Diego, CA (US); Mark Derdzinski, San Diego, CA (US); Sarah Kate Pickus, San Diego, CA (US); Lauren H. Jepson, San Diego, CA (US); Adam G. Noar, San Diego, CA (US); Douglas S. Kanter, San Diego, CA (US); Sonya Sokolash, San Diego, CA (US)

(73) Assignee: Dexcom, Inc., San Diego, CA (US)

(21) Appl. No.: 17/900,372

(22) Filed: Aug. 31, 2022

Related U.S. Application Data
(60) Provisional application No. 63/263,186, filed on Oct. 28, 2021.

(51) Int. Cl. A61B 5/145; A61B 5/00

(52) U.S. Cl. CPC A61B 5/14532; A61B 5/7475; A61B 5/742; A61B 5/002; A61B 2562/04

(57) ABSTRACT

Glucose monitoring over phases and corresponding phased information display is described. A multi-phase glucose monitoring program that includes at least a first phase and a second phase is initiated. First glucose data of a user is obtained during the first phase of the multi-phase glucose monitoring program. The output of the first glucose data in a glucose monitoring user interface is prevented during the first phase of the multi-phase glucose monitoring program. Second glucose data of the user is then obtained during a second phase of the multi-phase glucose monitoring program. The second glucose data is output, in real-time, in the glucose monitoring user interface during the second phase of the multi-phase glucose monitoring program.

### RELATED APPLICATION

This application claims the benefit of U.S. Provisional Patent Application No. 63/263,186, filed Oct. 28, 2021, and titled “Glucose Monitoring Over Phases and Corresponding Phased Information Display,” the entire disclosure of which is hereby incorporated by reference.

### BACKGROUND

Diabetes is a metabolic condition affecting hundreds of millions of people. For these people, monitoring blood glucose levels and regulating those levels to be within an acceptable range is important not only to mitigate long-term issues such as heart disease and vision loss, but also to avoid the effects of hyperglycemia and hypoglycemia. Maintaining blood glucose levels within an acceptable range can be challenging, as this level is almost constantly changing over time and in response to everyday events, such as eating or exercising. Advances in medical technologies have enabled development of various systems for monitoring blood glucose, including continuous glucose monitoring (CGM) systems, which measure and record glucose concentrations in substantially real-time. CGM systems are important tools for users of these systems to ensure that measured glucose values are within the acceptable range.

Glucose monitoring systems utilize wearable devices which include sensors that can be inserted into the skin of a user to monitor glucose and which are coupled to user devices (e.g., a user's smartphone) so that the glucose data can be output to the user, e.g., via a user interface. Oftentimes, however, users may become overwhelmed by the amount and/or complexity of the glucose data that is provided to the user. As a result, some users—particularly new users—may have a difficult time understanding their glucose data and thus fail to take meaningful actions to improve their health. Other users may alter their behavior in an attempt to control the output of glucose data. As an example, some users may eat food with lower carbohydrates in order to keep their glucose values within range. As a result, the glucose data captured by conventional CGM systems may not paint a realistic picture of the user's typical behavior and resulting glucose measurements.

### SUMMARY

To overcome these problems, glucose monitoring over phases and corresponding phased information display is leveraged. A multi-phase glucose monitoring program that includes at least a first phase and a second phase is initiated. First glucose data of a user is obtained during the first phase of the multi-phase glucose monitoring program. The output of the first glucose data in a glucose monitoring user interface is prevented during the first phase of the multi-phase glucose monitoring program. Second glucose data of the user is then obtained during a second phase of the multi-phase glucose monitoring program. The second glucose data is output, in real-time, in the glucose monitoring user interface during the second phase of the multi-phase glucose monitoring program.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an exemplary implementation that is operable to employ techniques described herein.

FIG. 2 depicts an example of the wearable glucose monitoring device of FIG. 1 in greater detail.

FIG. 3 depicts an example of a system to obtain glucose data of a person during a multi-phase glucose monitoring program and cause display of different respective information during the different phases.

FIG. 4 depicts an example implementation of a user interface in which output of glucose data, obtained during a phase of a multi-phase glucose monitoring program, is prevented.

FIG. 5 depicts an example implementation of the user interface displaying instructions for transitioning to a subsequent phase of the multi-phase glucose monitoring program.

FIG. 6 depicts an example implementation of the user interface displaying glucose data obtained during the subsequent phase of the multi-phase glucose monitoring program.

FIG. 7 depicts an example implementation of the user interface displaying user interface elements that are selectable to view more detailed information about one or more phases of the multi-phase glucose monitoring program.

FIG. 8 depicts an example implementation of the user interface displaying glucose data obtained during a subsequent phase of the multi-phase glucose monitoring program and displaying additional information related to the glucose data.

FIG. 9 depicts another example implementation of the user interface displaying glucose data obtained during a subsequent phase of the multi-phase glucose monitoring program and displaying additional information related to the glucose data.

FIG. 10 depicts a procedure in an example implementation of a multi-phase glucose monitoring program.

FIG. 11 depicts a procedure in an example implementation in which a glucose report is generated based on glucose data obtained during a first, second, third, and fourth phase of a multi-phase glucose monitoring program.

FIG. 12 illustrates an example of a system including various components of an example device that can be implemented as any type of computing device as described and/or utilized with reference to FIGS. 1-11 to implement embodiments of the techniques described herein.

### DETAILED DESCRIPTION

### Overview

Glucose monitoring over phases and corresponding phased information display is described. A multi-phase engine is configured to obtain glucose data (e.g., glucose measurements) over multiple phases of a glucose monitoring program. As an example, the multi-phase engine may obtain first glucose data during a first phase of a glucose monitoring program, obtain second glucose data during a second phase of the glucose monitoring program, obtain third glucose data during a third phase of the glucose monitoring program, and obtain fourth glucose data during a fourth phase of the glucose monitoring program. It should be appreciated that a multi-phase glucose monitoring program may have fewer phases (e.g., two) or more phases (e.g., five or more) without departing from the spirit or scope of the described techniques.

In one or more implementations, each phase utilizes a different wearable glucose sensor to obtain the glucose data. In this scenario, transitions between phases may correspond to the user replacing a glucose sensor associated with a previous phase with a new glucose sensor. In other words, the user wears a first glucose sensor during the first phase, and then replaces the first glucose sensor with a second glucose sensor in order to begin the second phase. It is to be appreciated, however, that a user may progress to different phases of a multi-phase glucose monitoring program based on the occurrence of different events or satisfaction of different conditions as discussed in more detail throughout.

In addition to obtaining glucose data, the multi-phase engine is configured to control display of information about the obtained glucose data during a multi-phase glucose monitoring program. In particular, the multi-phase engine controls display of the information such that different information is displayed during and following each phase of the program. In one or more implementations, for example, the multi-phase engine controls display of the information based on rules that define what information, if any, is displayed during and following each phase. As an example, the multi-phase engine can control what information related to the first, second, third, and fourth glucose data, respectively, is displayed during and between the first, second, third, and fourth phases of the multi-phase glucose monitoring program.

As part of determining what information is displayed during different phases, the multi-phase engine may be configured to determine an end of a phase. For example, the multi-phase engine may be configured in various ways to detect completion events indicating ends of phases. In relation to the first phase, for instance, the multi-phase engine may detect a completion event indicating completion of the first phase. Examples of completion events that may be detected by the multi-phase engine include detecting removal of a respective glucose sensor of the phase ending, detecting insertion of a glucose sensor for a phase subsequent to the phase ending, detecting expiration of a glucose monitoring time period associated with the phase ending, and detecting user input to terminate the phase that is ending and/or to initiate a next phase, to name just a few.

The multi-phase engine is configured to display information about the obtained glucose data via a user interface displayed on a display device of a computing device of the user, e.g., the user's smart watch or smartphone. In the first phase, for example, the multi-phase engine may prevent display via the user interface of the first glucose data obtained during the first phase. In other words, no glucose data is displayed via the user interface in the illustrated first phase. Doing so may encourage the user to “act normally” during the first phase, so that the user behaves in a similar manner as he or she would absent wearing the wearable glucose monitoring device. In this way, the first glucose data may provide a baseline against which the data of subsequent phases may be compared for deriving various insights about the user's behavior.

By way of contrast, the multi-phase engine may display via the user interface at least some of the second glucose data obtained during the second phase, e.g., a current glucose level of the user. Notably, the current glucose level of the user may correspond to information that the multi-phase engine prevented from being displayed during the first phase. Alternatively or additionally, the multi-phase engine may display via the user interface limited insights and/or limited recommendations derived from the second glucose data. It is to be appreciated that the information displayed at each phase may vary depending on a design (e.g., rules) of a given multi-phase glucose monitoring program. Broadly speaking, however, the multi-phase engine progressively reveals different information (e.g., more information and/or more detailed information) at subsequent phases of the glucose monitoring program. Similarly, in the third phase and/or the fourth phase of the glucose monitoring program, the multi-phase engine can control the user interface to display different information to the user, e.g., more information than the information displayed in the second phase.

Due to progressively revealing information, rather than presenting it to the user being monitored from the beginning of the program like conventional glucose monitoring systems, the multi-phase engine may learn how the user “normally” behaves—because information is not output causing the user to be “scared” into behaving in a way that is different from how he or she typically behaves. The multi-phase engine may also more effectively educate the user regarding what the information output via the user interface means because the person is only shown an amount of new information at each phase, such that he or she has time to learn about the information before additional and/or more detailed information is output at a subsequent phase. In other words, the multi-phase engine helps the person build upon his or her knowledge over time by progressively revealing information.

In the following discussion, an exemplary environment is first described that may employ the techniques described herein. Examples of implementation details and procedures are then described which may be performed in the exemplary environment as well as other environments. Performance of the exemplary procedures is not limited to the exemplary environment and the exemplary environment is not limited to performance of the exemplary procedures.

### Example of an Environment

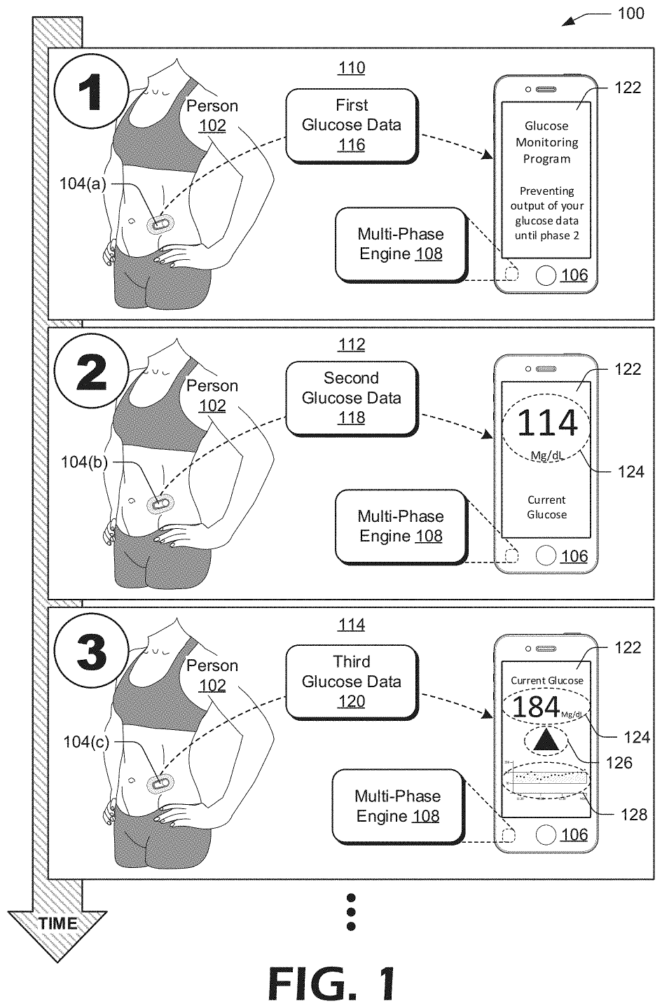

FIG. 1 is an illustration of an environment 100 in an example implementation that is operable to employ glucose monitoring over phases and corresponding phased information display as described herein. The illustrated environment 100 includes person 102, who is depicted wearing a wearable glucose monitoring device 104, examples of which include wearable glucose monitoring device 104(a), wearable glucose monitoring device 104(b), and wearable glucose monitoring device 104(c) having first, second, and third glucose sensors, respectively. The illustrated environment 100 also includes computing device 106, which is depicted having a multi-phase engine 108. The wearable glucose monitoring device 104 and the computing device 106 are communicatively coupled, including via a network (not shown).

The wearable glucose monitoring device 104 and the computing device 106 may be communicatively coupled in various ways, such as by using one or more wireless communication protocols or techniques. By way of example, the wearable glucose monitoring device 104 and the computing device 106 may communicate with one another using one or more of radio, cellular, Wi-Fi, Bluetooth (e.g., Bluetooth Low Energy links), near-field communication (NFC), 5G, and so forth.

In accordance with the described techniques, the wearable glucose monitoring device 104 is configured to provide measurements of the person 102's glucose. Although a wearable glucose monitoring device is discussed herein, it is to be appreciated that glucose monitoring over phases may be used with other devices capable of providing glucose measurements, e.g., non-wearable glucose devices (such as blood glucose meters requiring finger sticks), patches, and so forth. Additionally or alternatively, a multi-phase monitoring program may be used in connection with devices capable of producing measurements or making determinations about characteristics of the person 102 in addition to and/or different from glucose, e.g., temperature, other analytes (e.g., lactate, sodium, insulin, etc.), blood oxygen, heart rate, and heart rate variability, to name just a few. In implementations that involve the wearable glucose monitoring device 104, though, it may be configured with a glucose sensor that detects analytes indicative of the person 102's glucose and enables generation of glucose measurements as discussed above and below.

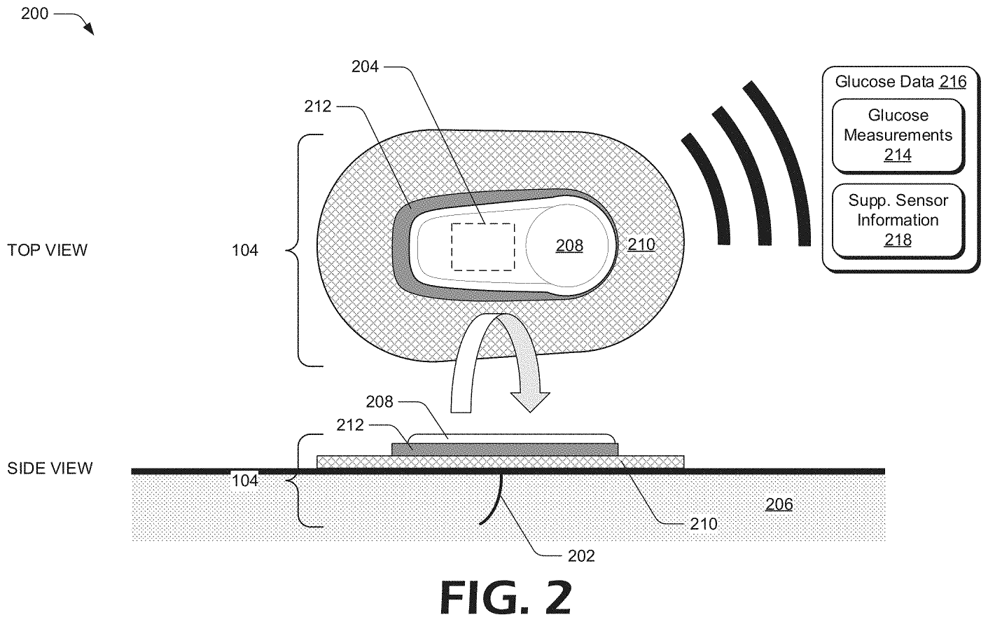

In one or more implementations, the wearable glucose monitoring device 104 is a continuous glucose monitoring (“CGM”) system. As used herein, the term “continuous” when used in connection with glucose monitoring may refer to an ability of a device to produce measurements substantially continuously, such that the device may be configured to produce the glucose measurements at regular or irregular intervals of time (e.g., approximately every hour, approximately every 30 minutes, approximately every 5 minutes, and so forth), responsive to establishing a communicative coupling with a different device (e.g., when the computing device 106 establishes a wireless connection with the wearable glucose monitoring device 104 to retrieve one or more of the measurements), and so forth. This functionality along with further aspects of the configuration of the wearable glucose monitoring device 104 are discussed in more detail in relation to FIG. 2.

Additionally, the wearable glucose monitoring device 104 transmits glucose measurements to the computing device 106, such as via a wireless connection. The wearable glucose monitoring device 104 may communicate these measurements in real-time, e.g., as they are produced using a glucose sensor. Alternatively or in addition, the wearable glucose monitoring device 104 may communicate the glucose measurements to the computing device 106 at set time intervals. For example, the wearable glucose monitoring device 104 may be configured to communicate the glucose measurements to the computing device 106 approximately every five minutes (as they are being produced). Certainly, an interval at which the glucose measurements are communicated may be different from the examples above without departing from the spirit or scope of the described techniques. The measurements may be communicated by the wearable glucose monitoring device 104 to the computing device 106 according to other bases in accordance with the described techniques, such as based on a request from the computing device 106. Regardless, the computing device 106 may maintain the glucose measurements of the person 102 at least temporarily, e.g., in computer-readable storage media of the computing device 106.

The computing device 106 may be configured in a variety of ways without departing from the spirit or scope of the described techniques. By way of example and not limitation, the computing device 106 may be configured as a mobile device (e.g., a mobile phone, a wearable device, or tablet device), a desktop computer, or a laptop computer, just to name a few form factors. In one or more implementations, the computing device 106 may be configured as a dedicated device associated with a glucose monitoring platform (not shown). A dedicated device associated with a glucose monitoring platform may be configured with functionality to obtain glucose measurements from the wearable glucose monitoring device 104, perform various computations in relation to the glucose measurements, display information related to the glucose measurements and the glucose monitoring platform, communicate the glucose measurements to the glucose monitoring platform, and so forth.

Additionally, the computing device 106 may be representative of more than one device in accordance with the described techniques. In one or more scenarios, for instance, the computing device 106 may correspond to both a wearable device (e.g., a smart watch) and a mobile phone. In such scenarios, both of these devices may be capable of performing at least some of the same operations, such as to receive glucose measurements from the wearable glucose monitoring device 104, communicate them via the network to a glucose monitoring platform, display information related to the glucose measurements, and so forth. Alternatively or in addition, different devices may have different capabilities that other devices do not have or that are limited through computing instructions to specified devices.

In the scenario where the computing device 106 corresponds to a separate smart watch and a mobile phone, for instance, the smart watch may be configured with various sensors and functionality to measure a variety of physiological markers (e.g., heartrate, heartrate variability, breathing, rate of blood flow, and so on) and activities (e.g., steps or other exercise) of the person 102. In this scenario, the mobile phone may not be configured with these sensors and functionality, or it may include a limited amount of that functionality—although in other scenarios a mobile phone may be able to provide the same functionality. Continuing with this particular scenario, the mobile phone may have capabilities that the smart watch does not have, such as a camera to capture images associated with glucose monitoring and an amount of computing resources (e.g., battery and processing speed) that enables the mobile phone to more efficiently carry out computations in relation to glucose measurements. Even in scenarios where a smart watch is capable of carrying out such computations, computing instructions may limit performance of those computations to the mobile phone so as not to burden both devices and to utilize available resources efficiently. To this extent, the computing device 106 may be configured in different ways and represent different numbers of devices than discussed herein without departing from the spirit and scope of the described techniques.

In accordance with the described techniques, the multi-phase engine 108 is configured to obtain glucose data (e.g., glucose measurements) over multiple phases of a monitoring program, e.g., multiple phases of a glucose monitoring program. The illustrated environment 100 depicts a first phase 110, a second phase 112, and a third phase 114 of a glucose monitoring program. It should be appreciated that a multi-phase monitoring program may have fewer phases (e.g., two) or more phases (e.g., four or more) without departing from the spirit or scope of the described techniques. Indeed, the ellipses depicted in the illustrated environment 100 below the third phase 114 indicate that such a program may include more phases. The illustrated environment 100 also includes an arrow to the left of the depicted phases indicating the passage of time, from top to bottom. In this example, therefore, the first phase 110 precedes the second phase 112 in time and the second phase 112 precedes the third phase 114 in time. The second phase 112 is thus subsequent to the first phase 110, and the third phase 114 is thus subsequent to the first and second phases 110, 112.

In environment 100, the multi-phase engine 108 obtains the first glucose data 116 during the first phase 110, obtains the second glucose data 118 during the second phase 112 and obtains the third glucose data 120 during the third phase 114. Here, the different phases each correspond to a different wearable glucose monitoring device 104, e.g., during the first phase 110 the first glucose data 116 is obtained from the wearable glucose monitoring device 104(a), during the second phase 112 the second glucose data 118 is obtained from the wearable glucose monitoring device 104(b), and during the third phase 114 the third glucose data 120 is obtained from the wearable glucose monitoring device 104(c). Although the different phases of the environment 100 each correspond to wear and use of a different wearable glucose monitoring device 104, it is to be appreciated that a user may progress to different phases of a multi-phase monitoring program based on the occurrence of different events or satisfaction of different conditions as discussed in more detail below.

In addition to obtaining glucose data, the multi-phase engine 108 is configured to control display of information about the obtained glucose data during a multi-phase glucose monitoring program. In particular, the multi-phase engine 108 controls display of the information such that different information is displayed during and following each phase of the program. In one or more implementations, for example, the multi-phase engine 108 controls display of the information based on rules that define what information, if any, is displayed during and following each phase. In the context of the illustrated environment 100, the multi-phase engine 108 controls what information related to the first, second, and third glucose data 116, 118, 120, respectively, is displayed during and between the first, second, and third phases 110, 112, 114 of the multi-phase monitoring program.

As part of determining what information is displayed during different phases, the multi-phase engine 108 may be configured to determine an end of a phase. For example, the multi-phase engine 108 may be configured in various ways to detect completion events indicating ends of phases. In relation to the first phase 110, for instance, the multi-phase engine 108 may detect a completion event indicating completion of the first phase 110. Examples of completion events that may be detected by the multi-phase engine 108 include detecting removal of a respective glucose sensor of the phase ending, detecting insertion of a glucose sensor for a phase subsequent to the phase ending, detecting expiration of a glucose monitoring time period associated with the phase ending, and detecting user input to terminate the phase that is ending and/or to initiate a next phase, to name just a few.

In the illustrated environment 100, the multi-phase engine 108 is configured to display information about the obtained glucose data via a user interface 122 displayed on a display device of the computing device 106. By way of example, the user interface 122 may correspond to an application downloaded to the computing device, e.g., an application associated with a multi-phase glucose monitoring program provided by a glucose monitoring platform. Alternatively or additionally, the user interface 122 may correspond to a different application, such as a browser application or third-party application. Regardless, the multi-phase engine 108 is configured to display different information via the user interface 122 for each of the different phases.

In the first phase 110, for example, the multi-phase engine 108 may prevent display via the user interface 122 of the first glucose data 116 obtained during the first phase 110—no glucose data is displayed via the user interface 122 in the illustrated first phase 110. One reason for preventing display of the first glucose data 116 may be to encourage the person 102 to “act normally” during the first phase 110, so that the person 102 behaves in a similar manner as he or she would absent wearing the wearable glucose monitoring device 104(a). In this way, the first glucose data 116 may provide a baseline against which the data of subsequent phases may be compared for deriving various insights about the person 102's behavior.

By way of contrast, the multi-phase engine 108 may display via the user interface 122 at least some of the second glucose data 118 obtained during the second phase 112, e.g., a current glucose level 124 of the person 102. In this example, the current glucose level 124 of the person 102 corresponds to information that the multi-phase engine 108 prevented from being displayed during the first phase 110. Alternatively or additionally, the multi-phase engine 108 may display via the user interface 122 limited insights and/or limited recommendations derived from the second glucose data 118. It is to be appreciated that the information displayed at each phase may vary depending on a design (e.g., rules) of a given multi-phase monitoring program. Broadly speaking, however, the multi-phase engine 108 progressively reveals different information (e.g., more information and/or more detailed information) at subsequent phases of a program.

In the illustrated third phase 114, the multi-phase engine 108 causes different information to be displayed via the user interface 122, e.g., more information than the information displayed in the second phase 112. Here, the user interface 122 is depicted including in the third phase 114, the current glucose level 124, a trend indicator 126, and a glucose trace 128. Notably, the multi-phase engine 108 prevented the trend indicator 126 and the glucose trace 128 from being presented in the first and second phases 110, 112 and then revealed that information in the third phase 114. Certainly, the multi-phase engine 108 may progressively reveal different information and varying amounts of information at each subsequent phase in a variety of ways without departing from the spirit or scope of the described techniques. In the context of measuring glucose, e.g., continuously, and obtaining glucose data describing such measurements, consider the following discussion of FIG. 2.

FIG. 2 depicts an example 200 of an implementation of the wearable glucose monitoring device 104 of FIG. 1 in greater detail. In particular, the illustrated example 200 includes a top view and a corresponding side view of the wearable glucose monitoring device 104. It is to be appreciated that the wearable glucose monitoring device 104 may vary in implementation from the following discussion in various ways without departing from the spirit or scope of the described techniques. As noted above, for instance, glucose monitoring over phases may be used in connection with other types of devices for glucose monitoring, such as non-wearable devices (e.g., blood glucose meters requiring finger sticks), patches, and so forth.

In this example 200, the wearable glucose monitoring device 104 is illustrated to include a glucose sensor 202 and a sensor module 204. Here, the glucose sensor 202 is depicted in the side view having been inserted subcutaneously into skin 206, e.g., of the person 102. The sensor module 204 is depicted in the top view as a dashed rectangle. The wearable glucose monitoring device 104 also includes a transmitter 208 in the illustrated example 200. Use of the dashed rectangle for the sensor module 204 indicates that it may be housed or otherwise implemented within a housing of the transmitter 208. In this example 200, the wearable glucose monitoring device 104 further includes adhesive pad 210 and attachment mechanism 212. In one or more implementations, the user may utilize a different sensor 202 and sensor module 204 for each of the different phases of the monitoring program. In these instances, however, the same transmitter 208 may be used for each of the different phases. Alternatively, a single sensor 202 may be utilized for multiple phases without the need to remove and insert a new sensor for each phase. In one or more implementations, the wearable glucose monitoring device 104 may be part of a sensor kit which includes a transmitter 208 and multiple different sensors 202 for each of the different phases. For example, the sensor kit may include one transmitter 208 and four different sensors 202 for each of the four phases of the glucose monitoring program.

In operation, the glucose sensor 202, the adhesive pad 210, and the attachment mechanism 212 may be assembled to form an application assembly, where the application assembly is configured to be applied to the skin 206 so that the glucose sensor 202 is subcutaneously inserted as depicted. In such scenarios, the transmitter 208 may be attached to the assembly after application to the skin 206 via the attachment mechanism 212. Alternatively, the transmitter 208 may be incorporated as part of the application assembly, such that the glucose sensor 202, the adhesive pad 210, the attachment mechanism 212, and the transmitter 208 (with the sensor module 204) can all be applied at once to the skin 206. In one or more implementations, this application assembly is applied to the skin 206 using a separate sensor applicator (not shown). Unlike the finger sticks required by conventional blood glucose meters, the user-initiated application of the wearable glucose monitoring device 104 is nearly painless and does not require the withdrawal of blood. Moreover, the automatic sensor applicator generally enables the person 102 to embed the glucose sensor 202 subcutaneously into the skin 206 without the assistance of a clinician or healthcare provider.

The application assembly may also be removed by peeling the adhesive pad 210 from the skin 206. It is to be appreciated that the wearable glucose monitoring device 104 and its various components as illustrated are simply one example form factor, and the wearable glucose monitoring device 104 and its components may have different form factors without departing from the spirit or scope of the described techniques.

In operation, the glucose sensor 202 is communicatively coupled to the sensor module 204 via at least one communication channel which can be a wireless connection or a wired connection. Communications from the glucose sensor 202 to the sensor module 204 or from the sensor module 204 to the glucose sensor 202 can be implemented actively or passively and these communications can be continuous (e.g., analog) or discrete (e.g., digital).

The glucose sensor 202 may be a device, a molecule, and/or a chemical which changes or causes a change in response to an event which is at least partially independent of the glucose sensor 202. The sensor module 204 is implemented to receive indications of changes to the glucose sensor 202 or caused by the glucose sensor 202. For example, the glucose sensor 202 can include glucose oxidase which reacts with glucose and oxygen to form hydrogen peroxide that is electrochemically detectable by the sensor module 204 which may include an electrode. In this example, the glucose sensor 202 may be configured as or include a glucose sensor configured to detect analytes in blood or interstitial fluid that are indicative of glucose level using one or more measurement techniques. In one or more implementations, the glucose sensor 202 may also be configured to detect analytes in the blood or the interstitial fluid that are indicative of other markers, such as lactate levels, which may improve accuracy in identifying or predicting glucose-based events. Additionally or alternatively, the wearable glucose monitoring device 104 may include additional sensors to the glucose sensor 202 to detect those analytes indicative of the other markers.

In another example, the glucose sensor 202 (or an additional sensor of the wearable glucose monitoring device 104—not shown) can include a first and second electrical conductor and the sensor module 204 can electrically detect changes in electric potential across the first and second electrical conductor of the glucose sensor 202. In this example, the sensor module 204 and the glucose sensor 202 are configured as a thermocouple such that the changes in electric potential correspond to temperature changes. In some examples, the sensor module 204 and the glucose sensor 202 are configured to detect a single analyte, e.g., glucose. In other examples, the sensor module 204 and the glucose sensor 202 are configured to detect multiple analytes, e.g., sodium, potassium, carbon dioxide, and glucose. Alternatively or additionally, the wearable glucose monitoring device 104 includes multiple sensors to detect not only one or more analytes (e.g., sodium, potassium, carbon dioxide, glucose, and insulin) but also one or more environmental conditions (e.g., temperature). Thus, the sensor module 204 and the glucose sensor 202 (as well as any additional sensors) may detect the presence of one or more analytes, the absence of one or more analytes, and/or changes in one or more environmental conditions.

In one or more implementations, the sensor module 204 may include a processor and memory (not shown). The sensor module 204, by leveraging the processor, may generate glucose measurements 214 based on the communications with the glucose sensor 202 that are indicative of the above-discussed changes. Based on the above-noted communications from the glucose sensor 202, the sensor module 204 is further configured to generate communicable packages of data that include at least one glucose measurement 214. In this example 200, glucose data 216 represents these packages of data. In one or more implementations, the first glucose data 116, the second glucose data 118, and the third glucose data 120 may include one or more such packets of the glucose data 216. Additionally or alternatively, the sensor module 204 may configure the glucose data 216 to include additional data, including, by way of example, supplemental sensor information 218. The supplemental sensor information 218 may include a sensor identifier, a sensor status, temperatures that correspond to the glucose measurements 214, measurements of other analytes that correspond to the glucose measurements 214, and so forth. It is to be appreciated that supplemental sensor information 218 may include a variety of data that supplements at least one glucose measurement 214 without departing from the spirit or scope of the described techniques.

In implementations where the wearable glucose monitoring device 104 is configured for wireless transmission, the transmitter 208 may transmit the glucose data 216 as a stream of data to a computing device. Alternatively or additionally, the sensor module 204 may buffer the glucose measurements 214 and/or the supplemental sensor information 218 (e.g., in memory of the sensor module 204 and/or other physical computer-readable storage media of the wearable glucose monitoring device 104) and cause the transmitter 208 to transmit the buffered glucose data 216 later at various regular or irregular intervals, e.g., time intervals (approximately every second, approximately every thirty seconds, approximately every minute, approximately every five minutes, approximately every hour, and so on), storage intervals (when the buffered glucose measurements 214 and/or supplemental sensor information 218 reach a threshold amount of data or a number of measurements), and so forth.

Having considered an example of an environment and an example of a wearable glucose monitoring device, consider now a discussion of some examples of details of the techniques for glucose monitoring over phases and phased information display in accordance with one or more implementations.

Glucose Monitoring Over Phases and Phased Information Display

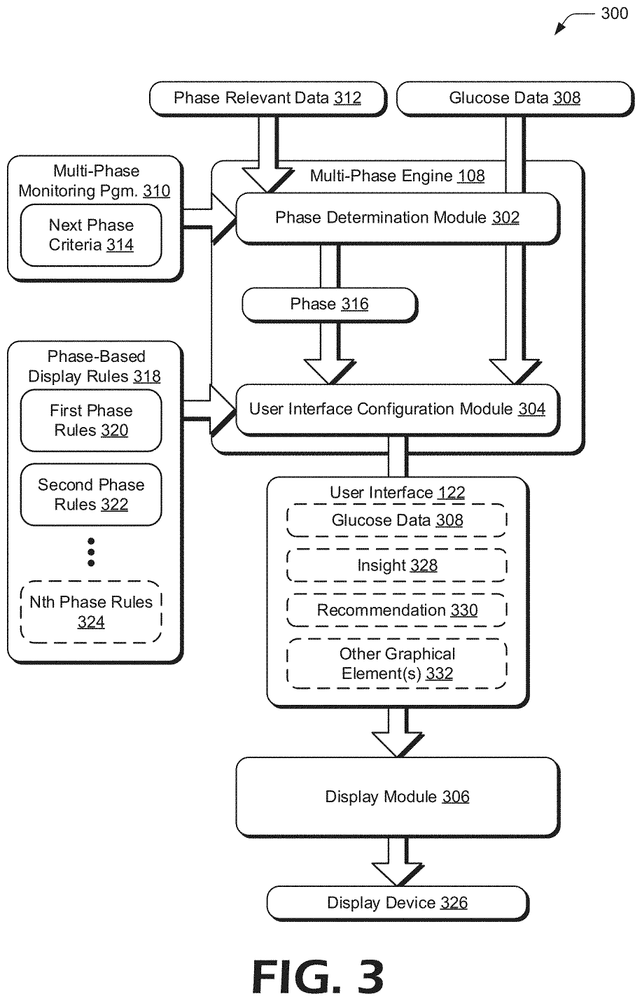

FIG. 3 depicts an example 300 of a system to obtain glucose data of a person during a multi-phase glucose monitoring program and cause display of different respective information during the different phases. The illustrated example 300 includes from FIG. 1 the multi-phase engine 108.

In example 300, the multi-phase engine 108 includes a phase determination module 302 and a user interface configuration module 304. The illustrated system also includes display module 306. In implementation, the system of example 300 may include more, fewer, or different components implementing a multi-phase monitoring program (e.g., a multi-phase glucose monitoring program) without departing from the spirit or scope of the described techniques. As noted above, the multi-phase engine 108 is configured to obtain glucose data during a multi-phase glucose monitoring program and also control display of information about the obtained glucose data during different phases of the program.

Here, the multi-phase engine 108 is depicted obtaining glucose data 308. By way of example, the glucose data 308 may correspond to the first glucose data 116 (e.g., during the first phase 110), the second glucose data 118 (e.g., during the second phase 112), the third glucose data 120 (e.g., during the third phase 114), or other glucose data of a respective phase.

The multi-phase engine 108 is also depicted obtaining a multi-phase monitoring program 310 and phase relevant data 312. In general, the multi-phase monitoring program 310 describes aspects of the program, which the multi-phase engine 108 uses to determine what, if any, information related to the glucose data 308 to output to a user (e.g., participating in the program), provide to a glucose monitoring platform (e.g., a service provider), and/or provide to other entities observing results of the program (e.g., physician or other health care provider, spouse, parent, or child). The multi-phase monitoring program 310 may also describe how users participating in the program may transition from one phase to a next, e.g., how a user may transition from the first phase 110 to the second phase 112.

In example 300, next phase criteria 314 corresponds to data describing how users in the multi-phase monitoring program 310 transition from one phase to a next. It is to be appreciated that users may transition to subsequent phases based on a variety of criteria, such as the occurrence or non-occurrence of one or more events, without departing from the spirit or scope of the described techniques. In one example, for instance, phases may be defined based on a glucose sensor worn by the person 102, such that when the person 102 changes from wearing a first sensor (e.g., included in the wearable glucose monitoring device 104(a)) to wearing a second sensor (e.g., included in the wearable glucose monitoring device 104(b)) the next phase criteria 314 describes that a next phase (e.g., the second phase 112) of the multi-phase monitoring program 310 begins. In other words, there is a one-to-one correspondence between glucose sensors worn (e.g., applied to the person 102's skin) during the multi-phase monitoring program and phases.

Certainly, transitions to subsequent phases may be based on criteria that are different from transitioning to wear different sensors. Rather than corresponding to a respective sensor, for instance, transitions to new phases may be based on behaviors of a user wearing one or more sensors. In these instances, a single sensor may be usable for multiple phases, e.g., for each of the multiple phases of the monitoring program. By way of example, transitions may correspond to the user performing one or more actions, e.g., performing an action a threshold number of times or according to an instructed pattern (a certain frequency or sequence). In one scenario, for instance, the next phase criteria 314 may define that a user transitions to a next phase of the multi-phase monitoring program 310 after performing an action a number of times. Examples of such an action may include capturing and uploading a photo (with the computing device 106) of a meal consumed by the user, accessing a computing application (or a user interface) associated with the multi-phase monitoring program 310, and providing input via a user interface to describe various aspects of the user's behavior or observations of conditions during the day (e.g., describing exercise, sleep, or stress)—in other words logging the user's behaviors and observations. Additional examples of such action may include going to sleep by a certain time (a number of days in a row or over the course of a phase), sleeping for a threshold number of hours a threshold number of days (in a row or over the course of a phase), taking a certain number of steps per day, and so forth. Certainly, the next phase criteria 314 may define different ways for transitioning to subsequent phases (e.g., “unlocking” a subsequent phase), including combinations of actions (e.g., logging behaviors and taking a number of steps), without departing from the spirit or scope of the described techniques.

In general, the phase relevant data 312 describes those actions, such that the described actions may be compared to the criteria to determine whether a user transitions to a subsequent phase of the multi-phase monitoring program 310. In accordance with the above-noted example actions that may cause transitions, for instance, the phase relevant data 312 may correspond to photos captured and uploaded by the user (e.g., of his or her meal), application data describing a number of times the user has accessed the computing application (or the user interface) associated with the multi-phase monitoring program 310, and/or saved data describing the various aspects of the user's behavior or observations of conditions during the day (based on the user input to enter such information). Alternatively or additionally, the phase relevant data 312 may correspond to tracked data describing the user's sleep and/or exercise, such as data collected from one or more devices (e.g., a smart watch). Indeed, the phase relevant data 312 may originate from a variety of sources and describe a variety of aspects of a user or environmental conditions that relate to transitioning between phases of the multi-phase monitoring program 310.

In this example 300, the multi-phase engine 108 includes phase determination module 302, which is configured to determine a phase 316 (e.g., a current phase) of the multi-phase monitoring program 310. The phase determination module 302 may determine the phase 316 by comparing the phase relevant data 312 to the next phase criteria 314. If the phase determination module 302 determines that the phase relevant data 312 satisfies the next phase criteria 314, then the phase determination module 302 determines that the phase 316 transitions to a next phase. If, however, the phase determination module 302 determines that the phase relevant data 312 does not satisfy the next phase criteria 314, then the phase determination module 302 determines that the phase 316 remains a same phase.

The multi-phase engine 108 also includes user interface configuration module 304. In accordance with the described techniques, the user interface configuration module 304 is configured to control output of information to a user participating in the multi-phase monitoring program 310 and control output of information to any other entity observing the multi-phase monitoring program 310 (e.g., communications to a health care provider or a glucose monitoring platform). In one or more implementations, for instance, the user interface configuration module 304 is configured to control the information output to the user participating in the multi-phase monitoring program 310 by configuring the user interface 122 to include information and/or by preventing the user interface 122 from including information. In accordance with the described techniques, for instance, the user interface configuration module 304 may prevent the user interface 122 from including first glucose data 116 during the first phase 110.

In one or more implementations, the user interface configuration module 304 uses phase-based display rules 318 to determine what information to display at each phase of the multi-phase monitoring program 310. Although depicted separately from the multi-phase monitoring program 310, the phase-based display rules 318 may included as part of or otherwise associated with the multi-phase monitoring program 310. Here, the phase-based display rules 318 are depicted having first phase rules 320, second phase rules 322, and Nth phase rules 324.

The first phase rules 320 specify which information about the glucose data 308, if any, is displayed during a first phase of a multi-phase monitoring program, e.g., the first phase 110. For instance, the first phase rules 320 specify the information that is allowed to be displayed during the first phase, and they may also specify the information that is not allowed to be displayed during the first phase. The user interface configuration module 304 prevents display of the information that the first phase rules 320 specify is not allowed to be displayed during the first phase. In the context of the first phase 110, for example, the first phase rules 320 may specify that glucose data is not to be displayed during the first phase 110. Based on such a rule, the user interface configuration module 304 prevents display of the first glucose data 116 during the first phase 110, e.g., via the user interface 122. In this way, the user interface configuration module 304 may filter the incoming data during a first phase of a program to selectively display varying amounts of the data incoming to the system, e.g., varying amounts (from none to substantially more than none) of the glucose data 308, the phase relevant data 312, and insights and/or recommendations that can be derived from that data.

In a similar manner, the second phase rules 322 specify which information about the glucose data 308, if any, is displayed during a second phase of a multi-phase monitoring program, e.g., the second phase 112. For instance, the second phase rules 322 specify the information that is allowed to be displayed during the second phase, and they may also specify the information that is not allowed to be displayed during the second phase. The user interface configuration module 304 prevents display of the information that the second phase rules 322 specify is not allowed to be displayed during the second phase. In the context of the second phase 112, for example, the second phase rules 322 may specify that glucose data is allowed to be displayed but recommendations and/or insights are not to be displayed during the second phase 112. Based on such a rule, the user interface configuration module 304 causes display of the second glucose data 118 and prevents display of the recommendations and/or insights during the second phase 112, e.g., via the user interface 122. In this way, the user interface configuration module 304 may filter the incoming data during a second phase of a program to selectively display varying amounts of the data incoming to the system, e.g., varying amounts (from none to substantially more than none) of the glucose data 308, the phase relevant data 312, and insights and/or recommendations that can be derived from that data.

It is to be appreciated that in accordance with the described techniques, a “multi-phase monitoring program” may only have two phases and thus only two sets of phase rules. When a multi-phase monitoring program, for instance, has only two phases the phase-based display rules 318 may only include the first phase rules 320 and the second phase rules 322. In this scenario, the phase-based display rules 318 may not include the Nth phase rules 324, e.g., third phase rules. However, when a multi-phase monitoring program does include more than two phases, the phase-based display rules 318 may include a respective set of rules for each phase. For example, when a multi-phase monitoring program includes three phases, the phase-based display rules 318 may include three sets of rules, e.g., the first phase rules 320, the second phase rules 322, and the Nth phase rules 324. When a multi-phase monitoring program includes four phases, the phase-based display rules 318 may include four sets of rules, e.g., the first phase rules 320, the second phase rules 322, third-phase rules, and the Nth phase rules 324. In the illustrated example, the Nth phase rules 324 are depicted with dashed lines to indicate that they are optional. In other words, one or more implementations may not include or otherwise use the Nth phase rules 324, e.g., when the multi-phase monitoring program 310 only includes two phases. The phase-based display rules 318 also include ellipses between the second phase rules 322 and the Nth phase rules 324, which indicates that there may be no additional sets of rules between the second phase rules 322 and the Nth phase rules 324 or there may be one or more additional set of rules between the second phase rules 322 and the Nth phase rules 324. Whether there are zero or a number of additional sets of rules, e.g., in addition to the first phase rules 320 and the second phase rules 322, may depend on a number of phases of the multi-phase monitoring program 310.

In general, the phase-based display rules 318 define what data is displayed during and between each phase of the multi-phase monitoring program 310 and the user interface configuration module 304 processes those rules during and between the phases, along with incoming information (e.g., the phase 316 and the glucose data 308), to determine which of the incoming information to incorporate as part of the user interface 122, which incoming information to prevent from being included as part of the user interface 122, and how to configure graphical elements of the user interface 122 to display the information that is allowed to be output.

In the illustrated example 300, the user interface configuration module 304 is depicted outputting the user interface 122 to display module 306, which is configured to cause display of the user interface 122 via a display device 326, e.g., a display device of the computing device 106 or some other display device. It is to be appreciated that the user interface 122 may be output in different or additional ways from display. For example, the user interface configuration module 304 may configure and cause the user interface to be output for communication over a network, e.g., for communication to a glucose monitoring platform, for communication to a portal for health care providers (telemedicine), and/or for communication via an email, to name just a few. Alternately or additionally, the user interface configuration module 304 may configure the user interface for audible output via a speaker, e.g., via a voice assistant device.

In this example, the user interface 122 includes the glucose data 308 (which may correspond to a respective phase's glucose data, such as the first glucose data first glucose data 116 during the first phase first phase 110), insight 328, recommendation 330, and other graphical elements 332. Each of the glucose data 308, the insight 328, the recommendation 330, and the other graphical elements 332 are illustrated with dashes. Those dashes indicate that the glucose data 308, the insight 328, the recommendation 330, and the other graphical elements 332 are optionally included as part of the user interface 122 at different phases of the multi-phase monitoring program 310. Their inclusion and exclusion from the user interface 122 depend on the phase-based display rules 318 specified for the different phases and the user interface configuration module 304's processing of those rules to generate the user interface 122 during and between the stages of the multi-phase monitoring program 310.

In general, the glucose data 308 may include or otherwise correspond to one or more glucose measurements produced by the wearable glucose monitoring device 104. For example, the glucose data 308 may be represented in the user interface 122 as one or more numerical values (e.g., a number representing a current glucose of the person 102) or as representations of those values (e.g., plotted indicators on a graph). The insight 328 comprises an inference or conclusion derived based on one or more statistics computed using the glucose data 308 (and/or the phase relevant data 312) and/or one or more predictions generated based on the glucose data 308 (and/or the phase relevant data 312).

Certainly, the user interface 122 may be configured by the user interface configuration module 304 to include any number of insights 328 in connection with a given phase, depending on the respective phase-based display rules 318. Examples of insights may include trends (e.g., the person's glucose is rising, lowering, or staying the same over a last number of measurements or over an amount of time; the person's blood pressure (or heart rate variability) is rising, lowering, or staying the same over an amount of time), an amount of time within or outside a range of glucose values over a previous time interval (e.g., “time-in-range”), a likelihood of some event occurring in the future (e.g., a glycemic event and/or the occurrence of a given glucose level), a relative change of some value or metric over time (e.g., a lowered average glucose, more time in range during a current phase than a previous one, and so on), predictions of values or metrics (e.g., A1C), and a relative change in a characteristic of a person (e.g., perceived increased or decreased stress, better or worse sleep), to name just a few.

The recommendation 330 comprises advice output for the person, such as to improve his or her data, maintain observed “healthy” data, and/or otherwise suggest a course of action (e.g., contact a health care provider). Like the insights 328, the user interface 122 may be configured by the user interface configuration module 304 to include any number of recommendations 330 in connection with a given phase, depending on the respective phase-based display rules 318. Examples of recommendations may include, for instance, suggestions related to activity (e.g., engaging in one or more exercises for a number of minutes per day or per week or taking a threshold number of steps per day), eating behaviors (e.g., specifying types of foods to eat, specifying foods not to eat, specifying amounts of foods to eat, specifying when to eat, and so on), sleeping behaviors (e.g., lying down for bed by at least a certain time or averaging a threshold number of hours of sleep per night), device-engagement behaviors (e.g., interacting with an app a threshold number of times, logging one or more behaviors a respective threshold number of times, reducing an amount of screen time, and so on), and medication behaviors (e.g., taking one or more medicines or supplements according to a schedule), to name just a few.

In one or more implementations, the user interface configuration module 304 may include or otherwise have access to an analytics engine (not shown) configured to generate the insights 328 and/or the recommendations 330. In implementations where such an analytics engine is maintained by a glucose monitoring platform and is configured as web-based service provider, the platform may have access to significantly more computing resources than the computing device 106. These resources may include numerous servers and include databases that store glucose- and health-data for a population of users, e.g., thousands or millions of hours of user data. This vast amount of data enables patterns (e.g., correspondences) to be identified across the population of users between glucose data and health data. Additionally, this amount of data enables identifiable patterns in the data to be relied on and distinguished from anomalies, which may appear as “normal” correspondences when a platform has access to only a small amount of data. Those patterns may enable the glucose data or the health data to be predicted with observation (input) of the other.

Moreover, a glucose monitoring platform may include the computing resources (e.g., storage and processing power) to deploy one or more machine learning models to “learn” those patterns (e.g., using one or more algorithms and historical) and to generate the predictions used to produce the insights 328 or the recommendations 330. Alternatively or additionally, such a glucose monitoring platform may build (e.g., train) one or more machine learning models using the data of the population and have the machine learning model as trained (or otherwise learned) loaded onto a remote device, such as the computing device 106. By way of example, the machine learning model as trained may be downloaded from the platform by the computing device 106. Then the computing device 106 may deploy the machine learning model—as trained (or otherwise learned) at the platform level—locally at the computing device to process data at the computing device 106. The multi-phase engine 108 may include or otherwise have access to a variety of machine learning models that generate information used for configuring the user interface 122 without departing from the spirit or scope of the described techniques.

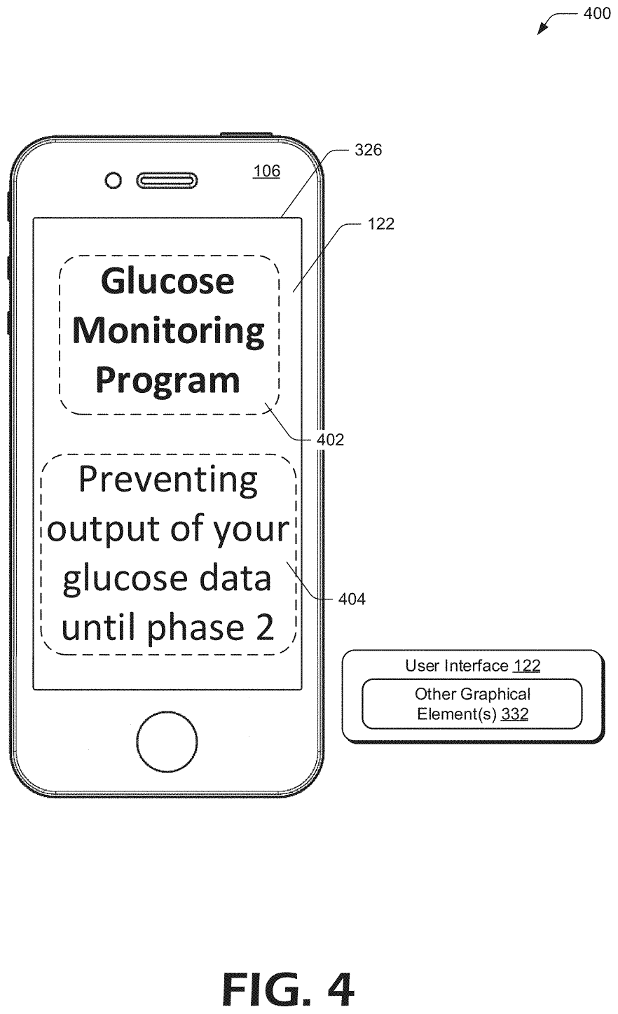

As noted above, the user interface 122 also includes other graphical elements 332. In general, the other graphical elements 332 comprise any of a variety of components that may be displayed as part of the user interface 122. Some examples of other graphical elements 332 include messages in text, text headings, menus, icons, buttons, images, videos, and so forth. Nonetheless, these other graphical elements 332 are distinct from the glucose data 308, the insights 328, and the recommendations 330, such that if the text of a message displayed via the user interface 122 includes the glucose data 308 it may not be considered one of the other graphical elements 332 but may instead be considered the glucose data 308. In the context of the information that may be included in user interfaces at different phases and information that may be prevented from being included in those user interfaces at the different phases, consider the following examples of user interfaces discussed in relation to FIGS. 4-9.

FIG. 4 depicts an example 400 of an implementation of a user interface in which output of glucose data, obtained during a phase of a multi-phase glucose monitoring program, is prevented.

The illustrated example 400 depicts the computing device 106 displaying the user interface 122 via the display device 326. Here, the user interface 122 is depicted presenting only the other graphical elements 332, namely, a label 402 (e.g., “Glucose Monitoring Program”) and message 404 (e.g., “Preventing output of your glucose data until phase 2”). The glucose data 308 is not included as part of the user interface 122. Rather, the user interface configuration module 304 prevents output of the glucose data 308 via the user interface 122. The user interface configuration module 304 may configure the user interface 122 in this way, for example, during a first phase of a multi-phase monitoring program, e.g., during the first phase 110 of the multi-phase glucose monitoring program of FIG. 1. In the context of FIG. 1, the user interface configuration module 304 thus prevents output of the first glucose data 116 during the first phase 110 by configuring the user interface 122 without the first glucose data 116 during the first phase 110.

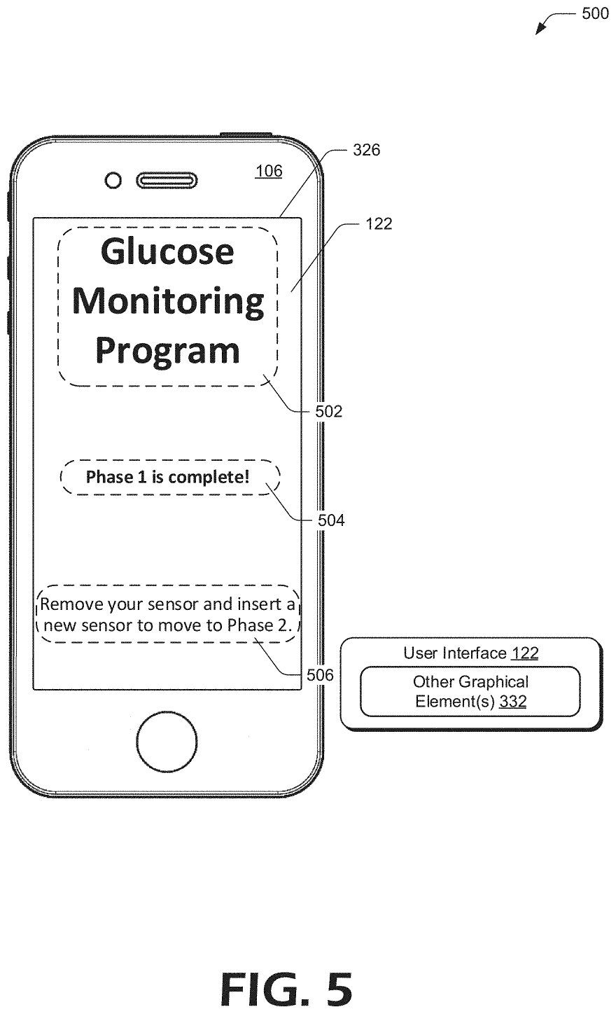

In one or more implementations, the user interface configuration module 304 may also prevent output of the first glucose data 116 between or during a transition period from the first phase 110 and the second phase 112, e.g., by configuring the user interface 122 during such a time period without the first glucose data 116. In the illustrated example 400, the user interface 122 also does not include any of the insights 328 or the recommendations 330. Thus, the user interface configuration module 304 may prevent display of the insights 328 and/or the recommendations 330 during the first phase 110 by not including them as part of the user interface 122. In the context of an example user interface that may be displayed between the first phase 110 and the second phase 112, consider the following discussion of FIG. 5.

FIG. 5 depicts an example 500 of an implementation of the user interface displaying instructions for transitioning to a subsequent phase of the multi-phase glucose monitoring program.

The illustrated example 500 depicts the computing device 106 displaying the user interface 122 via the display device 326. Here, the user interface 122 is again depicted presenting only the other graphical elements 332, namely, a label 502 (e.g., “Glucose Monitoring Program”), first message 504 (e.g., “Phase 1 is complete!”), and second message 506 (e.g., “Remove your sensor and insert a new sensor to move to Phase 2.”). In this example 500, the second message 506 comprises instructions that instruct a user, at least in part, regarding how to transition a subsequent phase of the multi-phase glucose monitoring program. Thus, the user interface configuration module 304 may continue to prevent output of one or more of the glucose data 308, the insights 328, and the recommendations 330 between the first phase 110 and the second phase 112. It is to be appreciated, however, that in different implementations, the user interface 122 may be configured to display different and/or additional information when transitioning between the first phase 110 and the second phase 112.

By way of example, the user interface configuration module 304 may configure the user interface 122 to include a button (e.g., other graphical element 332) that is selectable to display a report about the person 102's first phase 110 of the program, which may include one or more of the first glucose data 116, insights 328 derived based on the first glucose data 116, and/or recommendations 330 derived based on the first glucose data 116 and/or based on the insights 328. In such implementations, the user interface configuration module 304 therefore may not prevent the glucose data 308 (or the insights 328 and recommendations 330) from being displayed after the first phase first phase 110 and before the second phase 112. Whether the user interface configuration module 304 prevents the glucose data 308 from being displayed between phases (or during them) may depend on the phase-based display rules 318. In the context of a user interface that may be displayed during a phase subsequent to the first phase 110 of the multi-phase glucose monitoring program, consider the following example.

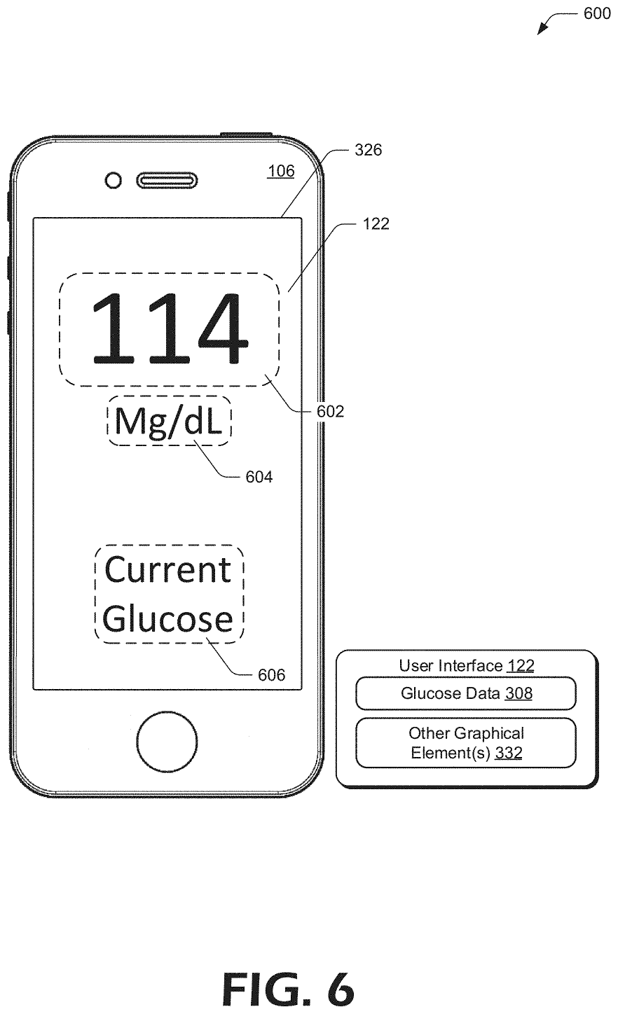

FIG. 6 depicts an example 600 of an implementation of the user interface displaying glucose data obtained during the subsequent phase of the multi-phase glucose monitoring program.

The illustrated example 600 depicts the computing device 106 displaying the user interface 122 via the display device 326. Here, the user interface 122 is depicted including the glucose data 308 and other graphical elements 332. In particular, the glucose data 308 displayed via the user interface 122 includes current glucose 602, and the other graphical elements 332 displayed via the user interface 122 include unit indicator 604 (e.g., “Mg/dL”) and value label 606, which indicates that the numerical value displayed is current glucose of a person—rather than corresponding to some other measurement, e.g., heartrate. In one or more implementations, the current glucose 602 is displayed by the user interface 122 in real-time, e.g., as the glucose data 308 is received from the wearable glucose monitoring device 104. In this way, the current glucose 602 may correspond to a most recently received glucose measurement—the glucose measurement most-recently produced by the wearable glucose monitoring device 104 and communicated off the wearable glucose monitoring device 104, e.g., to the computing device 106 via a wireless connection.

A user interface having this, or similar, information may be displayed during the second phase 112 of the multi-phase glucose monitoring program of FIG. 1. By configuring the user interface 122 substantially as depicted in this example 600 during the second phase 112 (e.g., by including the glucose data 308) after having configured the user interface 122 substantially as depicted in FIG. 4 during the first phase 110 (e.g., by preventing output of the glucose data 308), the user interface configuration module 304 may progressively reveal information obtained and determined during a multi-phase program.

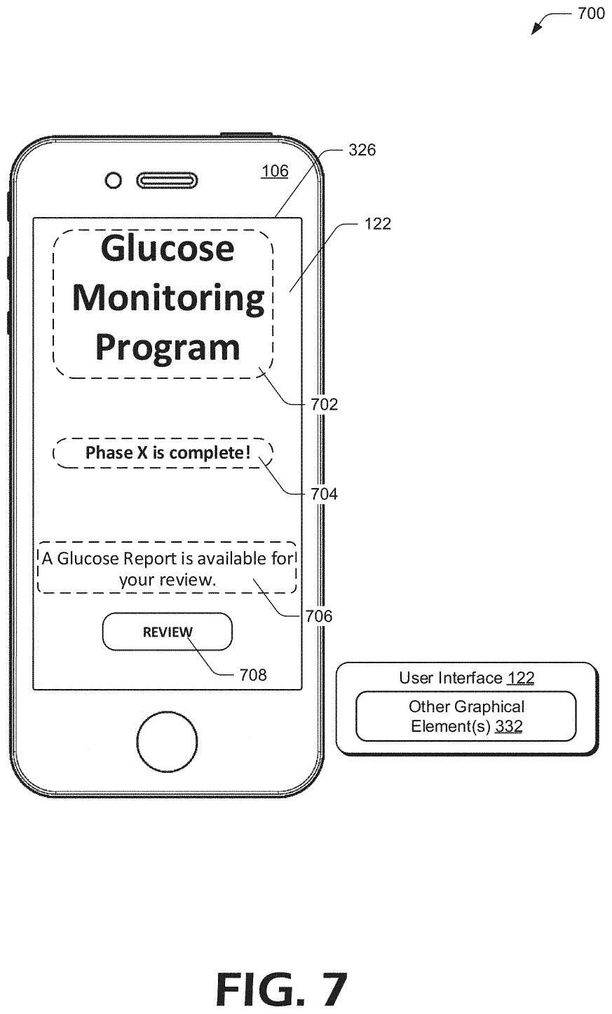

Due to progressively revealing information, rather than presenting it to the person 102 being monitored from the beginning of the program, the multi-phase engine 108 may learn how the person 102 “normally” behaves—because information is not output causing the person to be “scared” into behaving in a way that is different from how he or she typically behaves. The multi-phase engine 108 may also more effectively educate the person 102 regarding what the information output via the user interface 122 means because the person 102 is only shown an amount of new information at each phase, such that he or she has time to learn about the information before additional and/or more detailed information is output at a subsequent phase. In other words, the multi-phase engine 108 helps the person 102 build upon his or her knowledge over time by progressively revealing information. The multi-phase engine 108 may output or provide access to different information between phases than during them, such as aggregated information. In this context consider the following discussion of FIG. 7.

FIG. 7 depicts an example 700 of an implementation of the user interface displaying user interface elements that are selectable to view more detailed information about one or more phases of the multi-phase glucose monitoring program.

The illustrated example 700 depicts the computing device 106 displaying the user interface 122 via the display device 326. Here, the user interface 122 is depicted presenting other graphical elements 332, which include a label 702 (e.g., “Glucose Monitoring Program”), first message 704 (e.g., “Phase X is complete!”), and second message 706 (e.g., “A Glucose Report is available for your review.”). In this example 700, the other graphical elements 332 also include review button 708, which is selectable by a user of the computing device 106 to obtain a report of the person 102's glucose (e.g., over phase X). Thus, a report that may be presented responsive to selection of the review button 708 may display the glucose data 308. This contrasts with the example user interface of FIG. 5, which did not include functionality for accessing the glucose data 308. Like the user interfaces output during the phases, the user interfaces output following phases (or during the transitions to subsequent phases) may also progressively reveal information.

In this example 700, the “Glucose Report” may correspond to or otherwise include the glucose data 308 from at least the most-recently completed phase (e.g., Phase X). Additionally or alternatively, the “Glucose Report” may correspond to or otherwise include the glucose data 308 from one or more phases preceding the most-recently completed phase. In a scenario where “Phase X” corresponds to the second phase 112, for instance, a glucose report presented based on selection of the review button 708 may include at least some of the second glucose data 118 or may include at least some of the first glucose data 116 and the second glucose data 118. In one or more implementations, the glucose report may be generated based on the first glucose data 116 collected during the first phase 110 of the glucose monitoring program, the second glucose data 118 collected during the second phase 112 of the glucose monitoring program, the third glucose data 120 collected during the third phase 114 of the glucose monitoring program, and fourth glucose data collected during a fourth phase of the glucose monitoring program. Notably, therefore, even though glucose data collected during various phases may not be output during said phase (e.g., the first glucose data 116 may be prevented from being output during the first phase 110), this data can still be utilized and output at a later time, e.g., via the glucose report. The information included in such a report may be based, in part, on the information displayed during completed phases. For example, in a scenario where current glucose was displayed during a most recently completed phase, the multi-phase engine 108 may generate the report to include only the person's measured glucose over the course of the most recently completed phase (and/or any preceding phase). Indeed, the information displayed after completion of phases of a multi-phase monitoring program may vary in the spirit and scope of the described techniques.

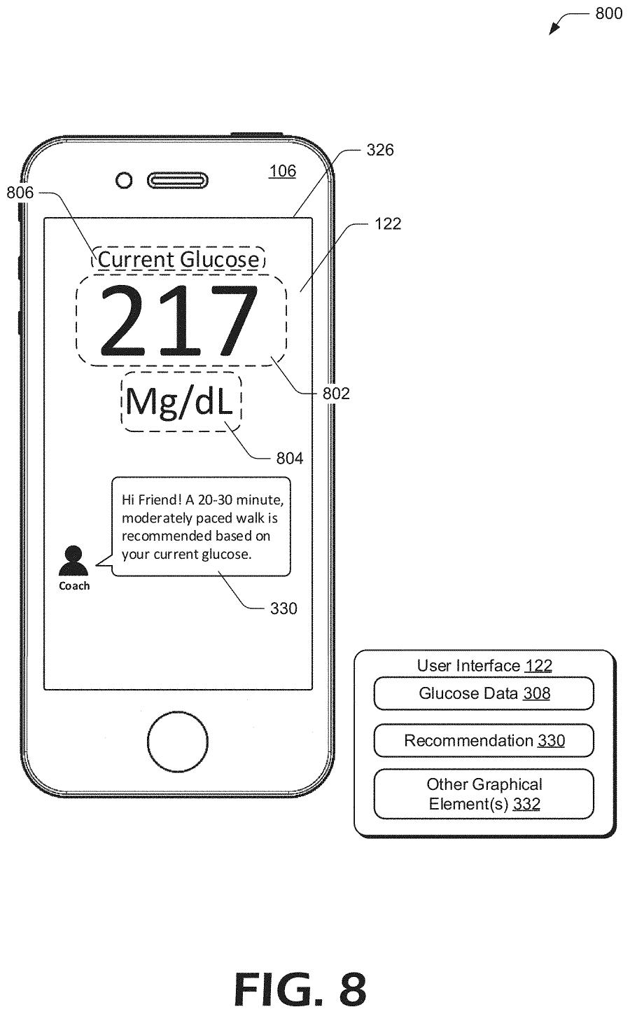

FIG. 8 depicts an example 800 of an implementation of the user interface displaying glucose data obtained during a subsequent phase of the multi-phase glucose monitoring program and displaying additional information related to the glucose data.

The illustrated example 800 depicts the computing device 106 displaying the user interface 122 via the display device 326. Here, the user interface 122 is depicted including the glucose data 308, the recommendation 330, and other graphical elements 332. In particular, the glucose data 308 displayed via the user interface 122 includes current glucose 802. In one or more implementations, the current glucose 802 is displayed by the user interface 122 in real-time, e.g., as the glucose data 308 is received from the wearable glucose monitoring device 104. In this way, the current glucose 802 may correspond to a most recently received glucose measurement—the glucose measurement most-recently produced by the wearable glucose monitoring device 104 and communicated off the wearable glucose monitoring device 104, e.g., to the computing device 106 via a wireless connection.

Like in FIG. 6, the other graphical elements 332 displayed via the user interface 122 may include unit indicator 804 (e.g., “Mg/dL”) and value label 806, which indicates that the numerical value displayed is current glucose of a person—rather than corresponding to some other measurement, e.g., heartrate. In contrast to FIG. 6 though, the user interface 122 is depicted displaying a recommendation 330.

The user interface 122 may be configured to include additional information (e.g., the recommendation 330 or an insight 328) along with the glucose data 308 at one or more times (e.g., phases) after a time (e.g., a phase) when the glucose data 308 is initially displayed. Alternatively, one or more recommendations 330 and/or insights 328 may be displayed via the user interface 122 before glucose data 308 is displayed. Thus, in some scenarios, the user interface 122 may be configured to include the glucose data 308 along with one or more recommendations 330 and/or insights 328 at one or more times (e.g., phases) after a time (e.g., phase) when a recommendation 330 and/or an insight 328 is initially displayed. The illustrated recommendation 330 is merely one example of a recommendation that may be presented to a person associated with a multi-phase monitoring program. Indeed, recommendations may be presented during a multi-phase monitoring program in a variety of manners in accordance with the described techniques.

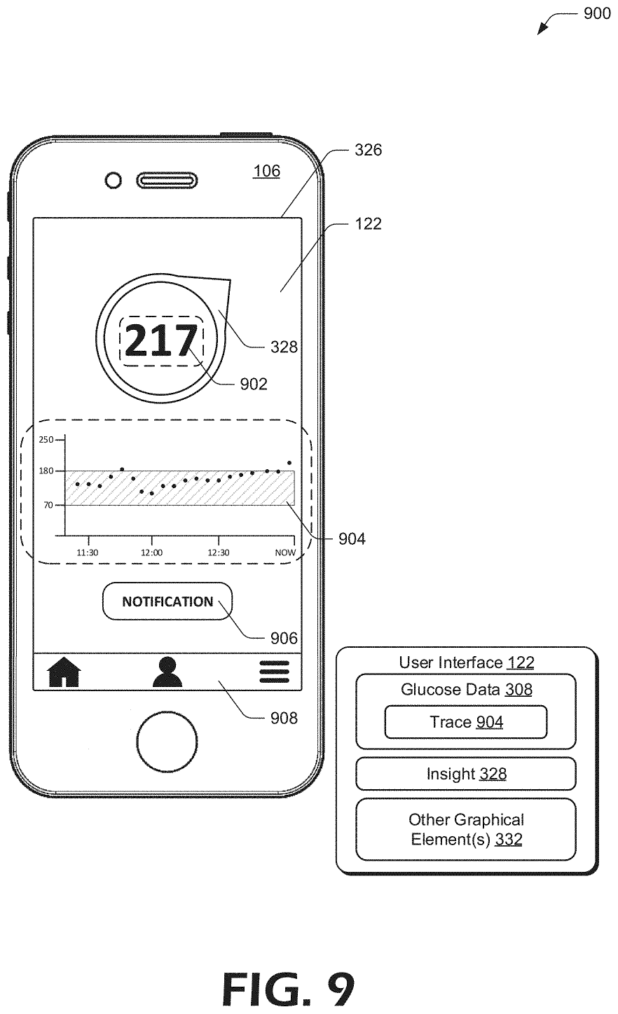

FIG. 9 depicts an example 900 of an implementation of the user interface displaying glucose data obtained during a subsequent phase of the multi-phase glucose monitoring program and displaying additional information related to the glucose data.

The illustrated example 900 depicts the computing device 106 displaying the user interface 122 via the display device 326. Here, the user interface 122 is depicted including the glucose data 308, the insight 328, and other graphical elements 332. In particular, the glucose data 308 displayed via the user interface 122 includes current glucose 902. As noted above, the current glucose 902 may be displayed by the user interface 122 in real-time, e.g., as the glucose data 308 is received from the wearable glucose monitoring device 104. In this way, the current glucose 902 may correspond to a most recently received glucose measurement—the glucose measurement most-recently produced by the wearable glucose monitoring device 104 and communicated off the wearable glucose monitoring device 104, e.g., to the computing device 106 via a wireless connection.

In addition to the current glucose 902, the glucose data 308 displayed via the user interface 122 also includes glucose trace 904 in this example 900. The insight 328 displayed in this example 900 is an indication of a trend, e.g., a graphical element indicating a trend of the person 102's glucose over time.

In the illustrated example 900, the indication of the trend surrounds the current glucose 902 and is generally circular with a triangular portion pointing in the direction of the trend, e.g., increasing glucose. For decreasing glucose, the triangle portion of the indication of the trend may point generally downward. Broadly speaking, the more rapidly glucose is observed to be increasing the more upward the triangular portion points and the more rapidly glucose is observed to be decreasing the more downward the triangular portion points. In scenarios where glucose remains substantially level, the trend indicator may point substantially horizontally. Certainly, the user interface 122 may be configured to present insights 328 different from indicators of glucose trends without departing from the spirit or scope of the described techniques. Additionally, an indicator of a glucose trend may indicate a trend in glucose in different ways from the depicted example.

In addition to the glucose data 308 (e.g., the current glucose 902 and the glucose trace 904), the user interface 122 also includes other graphical elements 332. In this example 900, the other graphical elements 332 include a notification button 906 and a menu 908 with various selectable options. The notification button 906 may be selectable by a user of the computing device 106 to present additional information from the illustrated example, such as an alert, more details about the displayed information, more insights 328, and recommendations 330 related to the information displayed, to name just a few. In accordance with the described techniques, the user interface 122 as depicted in the illustrated example 900 includes more information, generally, than the user interface as depicted in FIGS. 4-8. This indicates that the user interface 122 depicted in FIG. 9 corresponds to a later phase than the phases represented by the preceding examples. This further demonstrates that the user interfaces displayed in subsequent phases may include additional and/or more detailed information than is presented in preceding phases of a multi-phase monitoring program.

Having discussed exemplary details of the techniques for data-stream bridging for sensor transitions, consider now some examples of procedures to illustrate additional aspects of the techniques.

### Example Procedures

This section describes examples of procedures for glucose monitoring over phases and corresponding phased information display. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations the procedures are performed by a multi-phase engine, such as the multi-phase engine 108.

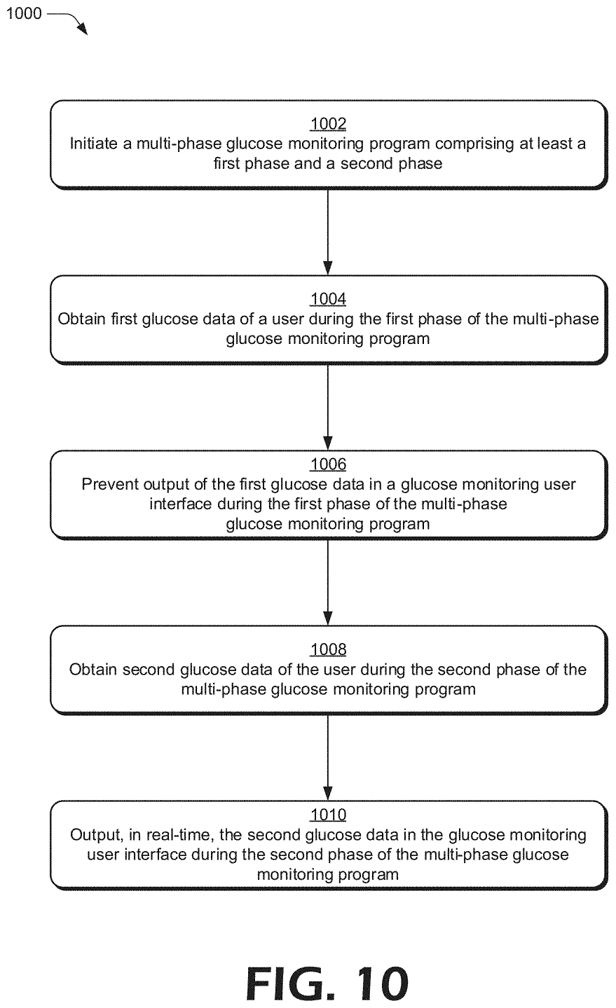

FIG. 10 depicts a procedure 1000 in an example implementation of a multi-phase glucose monitoring program.

A multi-phase glucose monitoring program that includes at least a first phase and a second phase is initiated (block 1002). By way of example, the multi-phase engine 108 initiates a multi-phase glucose monitoring program that includes at least a first phase 110 and a second phase 112.

First glucose data of a user is obtained during the first phase of the multi-phase glucose monitoring program (block 1004). By way of example, the multi-phase engine 108 obtains the first glucose data 116 during the first phase 110 of the glucose monitoring program.

Output of the first glucose data in a glucose monitoring user interface is prevented during the first phase of the multi-phase glucose monitoring program (block 1006). By way of example, in the first phase 110, the multi-phase engine 108 prevents, output of the first glucose data 116 obtained during the first phase 110 via the user interface 122. In other words, during the first phase 110, no glucose data is displayed via the user interface 122. One reason for preventing display of the first glucose data 116 may be to encourage the user to “act normally” during the first phase 110, so that the user behaves in a similar manner as he or she would absent wearing the wearable glucose monitoring device 104(a). In this way, the first glucose data 116 may provide a baseline against which the data of subsequent phases may be compared for deriving various insights about the user's behavior.

Second glucose data of the user is obtained during a second phase of the multi-phase glucose monitoring program (block 1008). By way of example, the multi-phase engine 108 obtains the second glucose data 118 during the second phase 112 of the glucose monitoring program.

The second glucose data is output, in real-time, in the glucose monitoring user interface during the second phase of the multi-phase glucose monitoring program (block 1010). By way of example, the multi-phase engine 108 may display via the user interface 122 at least some of the second glucose data 118 obtained during the second phase 112, e.g., a current glucose level 124 of the person 102. Notably, therefore, the second glucose data that is displayed during the second phase (e.g., the current glucose level 124 of the user) may correspond to information that the multi-phase engine 108 prevented from being displayed during the first phase 110. Alternatively or additionally, the multi-phase engine 108 may display via the user interface 122 limited insights and/or limited recommendations derived from the second glucose data 118.

Notably, the different phases may each correspond to a different wearable glucose monitoring device 104, e.g., during the first phase 110 the first glucose data 116 is obtained from the wearable glucose monitoring device 104(a), and during the second phase 112 the second glucose data 118 is obtained from the wearable glucose monitoring device 104(b). Although the different phases of the illustrated environment 100 each correspond to wear and use of a different wearable glucose monitoring device 104, it is to be appreciated that a user may progress to different phases of a multi-phase monitoring program based on the occurrence of different events or satisfaction of different conditions as discussed throughout.

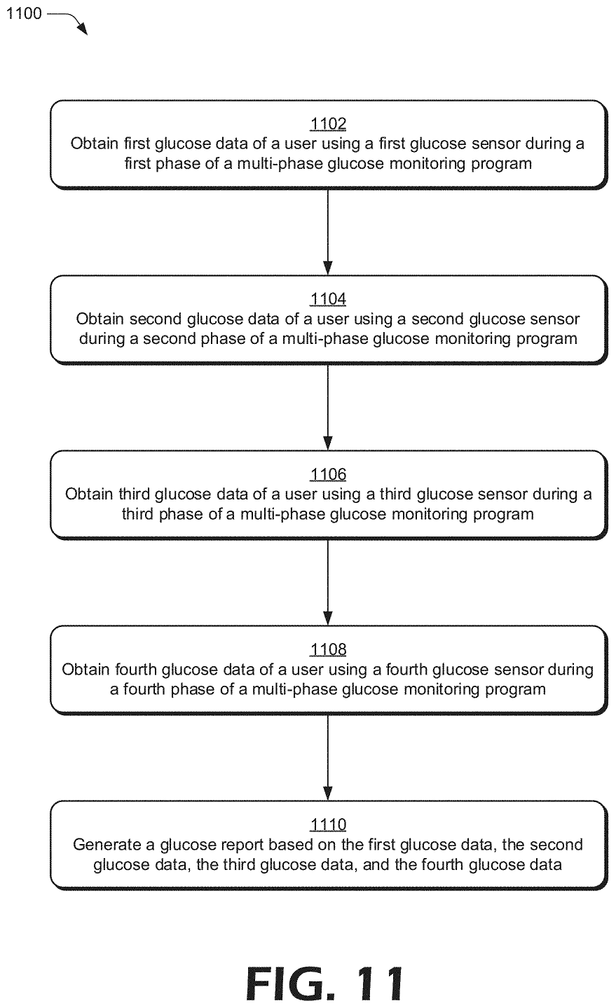

FIG. 11 depicts a procedure 1100 in an example implementation in which a glucose report is generated based on glucose data obtained during a first, second, third, and fourth phase of a multi-phase glucose monitoring program.

First glucose data of a user is obtained using a first glucose sensor during a first phase of a multi-phase glucose monitoring program (block 1102), second glucose data of the user is obtained using a second glucose sensor during a second phase of the multi-phase glucose monitoring program (block 1104), third glucose data of the user is obtained using a third glucose sensor during a third phase of the multi-phase glucose monitoring program (block 1106), and fourth glucose data of the user is obtained using a fourth glucose sensor during a fourth phase of the multi-phase glucose monitoring program (block 1108). By way of example, the multi-phase engine 108 obtains the first glucose data 116 during the first phase 110 of the glucose monitoring program, obtains the second glucose data 118 during the second phase 112 of the glucose monitoring program, obtains the third glucose data 120 during the third phase 114 of the glucose monitoring program, and obtains the fourth glucose data during the fourth phase of the glucose monitoring program.

A glucose report is generated based on the first glucose data, the second glucose data, the third glucose data, and the fourth glucose data (block 1110). By way of example, the multi-phase engine 108 generates the glucose report based on the first glucose data 116 collected during the first phase 110 of the glucose monitoring program, the second glucose data 118 collected during the second phase 112 of the glucose monitoring program, the third glucose data 120 collected during the third phase 114 of the glucose monitoring program, and fourth glucose data collected during a fourth phase of the glucose monitoring program. Notably, therefore, even though glucose data collected during various phases may not be output during said phase (e.g., the first glucose data 116 may be prevented from being output during the first phase 110), this data can still be utilized and output at a later time, e.g., via the glucose report.

Having described examples of procedures in accordance with one or more implementations, consider now an example of a system and device that can be utilized to implement the various techniques described herein.

### Example System and Device

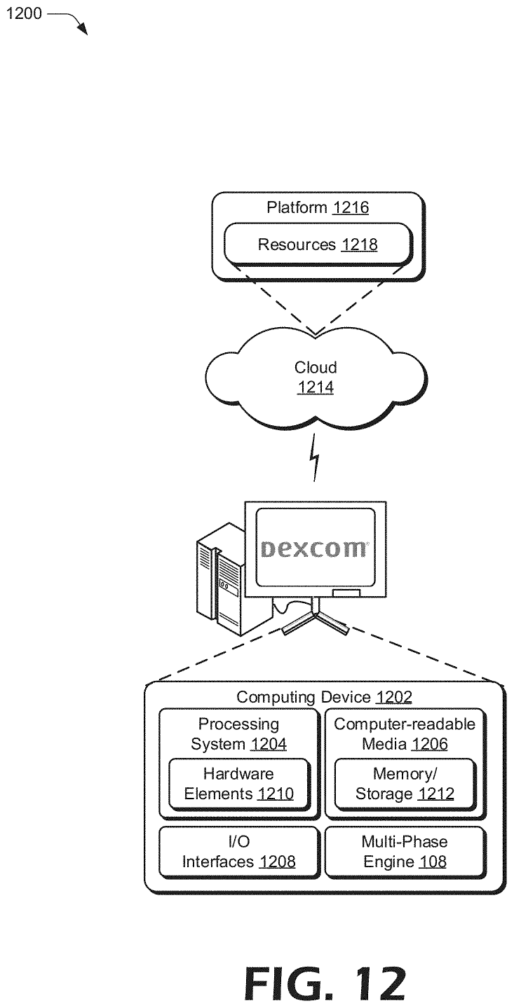

FIG. 12 illustrates an example of a system generally at 1200 that includes an example of a computing device 1202 that is representative of one or more computing systems and/or devices that may implement the various techniques described herein. This is illustrated through inclusion of the multi-phase engine 108. The computing device 1202 may be, for example, a server of a service provider, a device associated with a client (e.g., a client device), an on-chip system, and/or any other suitable computing device or computing system.

The example computing device 1202 as illustrated includes a processing system 1204, one or more computer-readable media 1206, and one or more I/O interfaces 1208 that are communicatively coupled, one to another. Although not shown, the computing device 1202 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1204 is representative of functionality to perform one or more operations using hardware. Accordingly, the processing system 1204 is illustrated as including hardware elements 1210 that may be configured as processors, functional blocks, and so forth. This may include implementation in hardware as an application specific integrated circuit or other logic device formed using one or more semiconductors. The hardware elements 1210 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 1206 is illustrated as including memory/storage 1212. The memory/storage 1212 represents memory/storage capacity associated with one or more computer-readable media. The memory/storage 1212 may include volatile media (such as random access memory (RAM)) and/or nonvolatile media (such as read only memory (ROM), Flash memory, optical disks, magnetic disks, and so forth). The memory/storage 1212 may include fixed media (e.g., RAM, ROM, a fixed hard drive, and so on) as well as removable media (e.g., Flash memory, a removable hard drive, an optical disc, and so forth). The computer-readable media 1206 may be configured in a variety of other ways as further described below.

Input/output interface(s) 1208 are representative of functionality to allow a user to enter commands and information to computing device 1202, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1202 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 1202. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1202, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1210 and computer-readable media 1206 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1210. The computing device 1202 may be configured to implement particular instructions and/or functions corresponding to the software and/or hardware modules. Accordingly, implementation of a module that is executable by the computing device 1202 as software may be achieved at least partially in hardware, e.g., through use of computer-readable storage media and/or hardware elements 1210 of the processing system 1204. The instructions and/or functions may be executable/operable by one or more articles of manufacture (for example, one or more computing devices 1202 and/or processing systems 1204) to implement techniques, modules, and examples described herein.

The techniques described herein may be supported by various configurations of the computing device 1202 and are not limited to the specific examples of the techniques described herein. This functionality may also be implemented all or in part through use of a distributed system, such as over a “cloud” 1214 via a platform 1216 as described below.

The cloud 1214 includes and/or is representative of a platform 1216 for resources 1218. The platform 1216 abstracts underlying functionality of hardware (e.g., servers) and software resources of the cloud 1214. The resources 1218 may include applications and/or data that can be utilized while computer processing is executed on servers that are remote from the computing device 1202. Resources 1218 can also include services provided over the Internet and/or through a subscriber network, such as a cellular or Wi-Fi network.

The platform 1216 may abstract resources and functions to connect the computing device 1202 with other computing devices. The platform 1216 may also serve to abstract scaling of resources to provide a corresponding level of scale to encountered demand for the resources 1218 that are implemented via the platform 1216. Accordingly, in an interconnected device embodiment, implementation of functionality described herein may be distributed throughout the system 1200. For example, the functionality may be implemented in part on the computing device 1202 as well as via the platform 1216 that abstracts the functionality of the cloud 1214.

### CONCLUSION

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. A computer-implemented method comprising:
initiating a multi-phase glucose monitoring program comprising at least a first phase and a second phase;
obtaining first glucose data of a user during the first phase of the multi-phase glucose monitoring program;
preventing output of the first glucose data in a glucose monitoring user interface during the first phase of the multi-phase glucose monitoring program;
obtaining second glucose data of the user during the second phase of the multi-phase glucose monitoring program; and
outputting, in real-time, the second glucose data in the glucose monitoring user interface during the second phase of the multi-phase glucose monitoring program.

2. The computer-implemented method as described in claim 1, wherein the first glucose data is collected by a first glucose sensor worn by the user during the first phase of the multi-phase glucose monitoring program and the second glucose data is collected by a second glucose sensor worn by the user during the second phase of the multi-phase glucose monitoring program.

3. The computer-implemented method as described in claim 2, wherein each of the first glucose data and the second glucose data is obtained from a single transmitter that is communicatively coupled to a computing device that displays the glucose monitoring user interface.

4. The computer-implemented method as described in claim 2, further comprising detecting a completion event indicating completion of the first phase.

5. The computer-implemented method as described in claim 4, further comprising outputting information based on the first glucose data in the glucose monitoring user interface after detecting the completion of the first phase.

6. The computer-implemented method as described in claim 4, wherein the completion event comprises one of:
detecting a removal of the first glucose sensor worn by the user during the first phase;
detecting an insertion of the second glucose sensor for the second phase;
detecting an expiration of a glucose monitoring time period associated with the first phase of the multi-phase glucose monitoring program; or
detecting user input, via the glucose monitoring user interface, to terminate the first phase or initiate the second phase.

7. The computer-implemented method as described in claim 1, wherein the first glucose data and the second glucose data is collected by a single glucose sensor.

8. The computer-implemented method as described in claim 1, further comprising generating a glucose report for the user at completion of the multi-phase glucose monitoring program based at least in part on the first glucose data and the second glucose data.

9. A computer-implemented method comprising:
obtaining first glucose data of a user using a first glucose sensor during a first phase of a multi-phase glucose monitoring program;
obtaining second glucose data of the user using a second glucose sensor during a second phase of the multi-phase glucose monitoring program;
obtaining third glucose data of the user using a third glucose sensor during a third phase of the multi-phase glucose monitoring program;
obtaining fourth glucose data of the user using a fourth glucose sensor during a fourth phase of the multi-phase glucose monitoring program; and
generating a glucose report based on the first glucose data, the second glucose data, the third glucose data, and the fourth glucose data.

10. The computer-implemented method as described in claim 9, further comprising controlling a glucose monitoring user interface during the multi-phase glucose monitoring program by progressively revealing information based on the first glucose data, the second glucose data, the third glucose data, and the fourth glucose data.

11. The computer-implemented method as described in claim 10, wherein the controlling the glucose monitoring user interface during the multi-phase glucose monitoring program further comprises:
preventing output of the first glucose data in a first configuration of the glucose monitoring user interface during the first phase of the multi-phase glucose monitoring program;
outputting, in real-time, the second glucose data in a second configuration of the glucose monitoring user interface during the second phase of the multi-phase glucose monitoring program;
outputting, in real-time, the third glucose data in a third configuration of the glucose monitoring user interface along with one or more glucose insights or recommendations during the third phase of the multi-phase glucose monitoring program; and
outputting, in real-time, the fourth glucose data in a fourth configuration of the glucose monitoring user interface along with one or more additional glucose insights or recommendations during the fourth phase of the multi-phase glucose monitoring program.

12. The computer-implemented method of claim 11, further comprising generating a baseline based on the first glucose data obtained during the first phase of the multi-phase glucose monitoring program, wherein the one or more glucose insights or the one or more additional glucose insights are generated based at least in part on the baseline.

13. The computer-implemented method of claim 10, wherein each of the first glucose data, the second glucose data, the third glucose data, and the fourth glucose data is obtained from a single transmitter that is communicatively coupled to a computing device that displays the glucose monitoring user interface.

14. The computer-implemented method of claim 13, wherein the single transmitter is coupled to the first glucose sensor, the second glucose sensor, the third glucose sensor, and the fourth glucose sensor during the first phase, the second phase, the third phase, and the fourth phase, respectively.

15. A system comprising:
a sensor kit for a multi-phase glucose monitoring program, the sensor kit comprising at least a first glucose sensor for monitoring glucose during a first phase of the multi-phase glucose monitoring program and a second glucose sensor for monitoring glucose during a second phase of the multi-phase glucose monitoring program; and
a multi-phase engine configured to:
control a glucose monitoring user interface during the multi-phase glucose monitoring program;
obtain first glucose data of a user during the first phase of the multi-phase glucose monitoring program from the first glucose sensor of the sensor kit;
prevent output of the first glucose data in the glucose monitoring user interface during the first phase of the multi-phase glucose monitoring program;
obtain second glucose data of the user during the second phase of the multi-phase glucose monitoring program; and
output, in real-time, the second glucose data in the glucose monitoring user interface during the second phase of the multi-phase glucose monitoring program.

16. The system of claim 15, wherein the sensor kit further comprises a third glucose sensor for monitoring glucose during a third phase of the multi-phase glucose monitoring program and a fourth glucose sensor for monitoring glucose during a fourth phase of the multi-phase glucose monitoring program.

17. The system of claim 16, wherein the multi-phase engine is further configured to:
obtain third glucose data of the user during the third phase of the multi-phase glucose monitoring program from the third glucose sensor of the sensor kit;
output, in real-time, the third glucose data in the glucose monitoring user interface along with one or more glucose insights or recommendations during the third phase of the multi-phase glucose monitoring program;
obtain fourth glucose data of the user during the fourth phase of the multi-phase glucose monitoring program; and
output, in real-time, the fourth glucose data in the glucose monitoring user interface along with one or more additional glucose insights or recommendations during the fourth phase of the multi-phase glucose monitoring program.

18. The system of claim 17, further comprising a transmitter configured to form a wireless connection with a computing device that display the glucose monitoring user interface, the transmitter configured to communicate the first glucose data, the second glucose data, the third glucose data, and the fourth glucose data to the computing device.

19. A computer-readable storage device comprising instructions stored thereon that, responsive to execution by one or more processors, performs operations comprising:
initiating a multi-phase glucose monitoring program comprising at least a first phase and a second phase;
obtaining first glucose data of a user during the first phase of the multi-phase glucose monitoring program;
preventing output of the first glucose data in a glucose monitoring user interface during the first phase of the multi-phase glucose monitoring program;
obtaining second glucose data of the user during a second phase of the multi-phase glucose monitoring program; and
outputting, in real-time, the second glucose data in the glucose monitoring user interface during the second phase of the multi-phase glucose monitoring program.

20. The computer-readable storage device as described in claim 19, wherein the first glucose data is collected by a first glucose sensor worn by the user during the first phase of the multi-phase glucose monitoring program and the second glucose data is collected by a second glucose sensor worn by the user during the second phase of the multi-phase glucose monitoring program.
